(19) United States
(12) Patent Application Publication
(10) Pub. No.: US 2025/0345257 A1
(43) Pub. Date: Nov. 13, 2025

(54) USE OF SACCHARIDES FOR CRYOPROTECTION AND RELATED TECHNOLOGY

(71) Applicant: Zeltiq Aesthetics, Inc., North Chicago, IL (US)

(72) Inventors: Joel N. Jimenez Lozano, Dublin, CA (US); Leonard C. DeBenedictis, Dublin, CA (US); Like Zeng, North Chicago, IL (US); George Frangineas, JR., Fremont, CA (US)

(21) Appl. No.: 19/171,011

(22) Filed: Apr. 4, 2025

Related U.S. Application Data
(63) Continuation of application No. 18/595,026, filed on Mar. 4, 2024, now abandoned.
Application No. 18/595,026 is a continuation of application No. 18/092,928, filed on Jan. 3, 2023, now abandoned.
Application No. 18/092,928 is a continuation of application No. 15/914,840, filed on Mar. 7, 2018, now abandoned.
(60) Provisional application No. 62/474,508, filed on Mar. 21, 2017.

(51) Int. Cl. A61K 8/60; A61B 18/00; A61B 18/02; A61B 90/00; A61F 7/00; A61K 8/36; A61K 8/42; A61K 8/86; A61N 1/30; A61N 1/32; A61N 7/00; C07H 3/04

(52) U.S. Cl. CPC A61K 8/602; A61B 18/0206; A61F 7/007; A61K 8/361; A61K 8/42; A61K 8/86; A61N 1/30; A61N 1/327; C07H 3/04; A61B 2018/00291; A61B 2018/00464; A61B 2018/0237; A61B 2018/0262; A61B 2090/0463; A61F 2007/0052; A61F 2007/0056; A61F 2007/0076; A61F 2007/0087; A61F 2007/0093; A61F 2007/0094; A61K 2800/244; A61N 2007/0034

(57) ABSTRACT

A method in accordance with a particular embodiment of the present invention includes increasing a concentration of a modified or unmodified saccharide within a subject's skin, applying an applicator to the subject's skin, and cooling the subject's skin via a heat-transfer surface of the applicator. The saccharide within the subject's skin can enhance a resistance of at least some cells within the subject's skin to damage associated with the cooling. A corresponding system includes the applicator, the saccharide, and an energy-delivery device. The energy-delivery device can be configured to apply ultrasound, optical, thermal, or another type of energy to the subject's skin to drive the saccharide into the subject's skin. The system can also include a penetration enhancer configured to enhance penetration of the saccharide into the subject's skin. The penetration enhancer can be applied with the saccharide or separately.

### CROSS-REFERENCE TO RELATED APPLICATION

The present application is a continuation application of U.S. patent application Ser. No. 15/914,480, filed Mar. 7, 2018, which claims the benefit of the earlier filing date of U.S. Provisional Patent Application No. 62/474,508, filed Mar. 21, 2017, the contents of each are incorporated herein by reference in their entirety.

### TECHNICAL FIELD

The present disclosure is related to cooling tissue, such as in the context of cryolipolysis and cryolysis.

### INCORPORATION BY REFERENCE

The following commonly assigned U.S. patent applications and U.S. patents are incorporated herein by reference in their entireties:
U.S. Pat. No. 7,854,754 entitled “COOLING DEVICE FOR REMOVING HEAT FROM SUBCUTANEOUS LIPID-RICH CELLS”;
U.S. Pat. No. 8,337,539 entitled “COOLING DEVICE FOR REMOVING HEAT FROM SUBCUTANEOUS LIPID-RICH CELLS”;
U.S. Patent Publication No. 2013/0158636 entitled “COOLING DEVICE FOR REMOVING HEAT FROM SUBCUTANEOUS LIPID-RICH CELLS”;
U.S. Pat. No. 8,192,474 entitled “TISSUE TREATMENT METHODS”;
U.S. Patent Publication No. 2013/0066309 entitled “TISSUE TREATMENT METHODS”;
U.S. Patent Publication No. 2015/0328077 entitled “TISSUE TREATMENT METHODS”;
U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;
U.S. Pat. No. 9,375,345 entitled “COOLING DEVICE HAVING A PLURALITY OF CONTROLLABLE COOLING ELEMENTS TO PROVIDE A PREDETERMINED COOLING PROFILE”;
U.S. Publication No. 2015/0342780 entitled “COOLING DEVICE HAVING A PLURALITY OF CONTROLLABLE COOLING ELEMENTS TO PROVIDE A PREDETERMINED COOLING PROFILE”;
U.S. Patent Publication No. 2008/0077201 entitled “COOLING DEVICES WITH FLEXIBLE SENSORS”;
U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;
U.S. Patent Publication No. 2014/0005760 entitled “CRYOPROTECTANT FOR USE WITH A TREATMENT DEVICE FOR IMPROVED COOLING OF SUBCUTANEOUS LIPID-RICH CELLS”;
U.S. Patent Publication No. 2007/0270925 entitled “METHOD AND APPARATUS FOR NON-INVASIVELY REMOVING HEAT FROM SUBCUTANEOUS LIPID RICH CELLS INCLUDING A COOLANT HAVING A PHASE TRANSITION TEMPERATURE”;
U.S. Patent Publication No. 2009/0118722 entitled “METHOD AND APPARATUS FOR COOLING SUBCUTANEOUS LIPID-RICH CELLS OR TISSUE”;
U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;
U.S. Patent Publication No. 2013/0079684 entitled “METHOD OF ENHANCED REMOVAL OF HEAT FROM SUBCUTANEOUS LIPID-RICH CELLS AND TREATMENT APPARATUS HAVING AN ACTUATOR”;
U.S. Pat. No. 8,285,390 entitled “MONITORING THE COOLING OF SUBCUTANEOUS LIPID-RICH CELLS, SUCH AS THE COOLING OF ADIPOSE TISSUE”;
U.S. Pat. No. 9,408,745 entitled “MONITORING THE COOLING OF SUBCUTANEOUS LIPID-RICH CELLS, SUCH AS THE COOLING OF ADIPOSE TISSUE”;
U.S. Pat. No. 9,408,745 entitled “MONITORING THE COOLING OF SUBCUTANEOUS LIPID-RICH CELLS, SUCH AS THE COOLING OF ADIPOSE TISSUE”;
U.S. Pat. No. 8,523,927 entitled “SYSTEM FOR TREATING LIPID-RICH REGIONS”;
U.S. Pat. No. 9,655,770 entitled “SYSTEM FOR TREATING LIPID-RICH REGIONS”;
U.S. Patent Publication No. 2009/0018624 entitled “LIMITING USE OF DISPOSABLE SYSTEM PATIENT PROTECTION DEVICES”;
U.S. Patent Publication No. 2009/0018625 entitled “MANAGING SYSTEM TEMPERATURE TO REMOVE HEAT FROM LIPID-RICH REGIONS”;
U.S. Patent Publication No. 2009/0018626 entitled “USER INTERFACES FOR A SYSTEM THAT REMOVES HEAT FROM LIPID-RICH REGIONS”;
U.S. Patent Publication No. 2009/0018627 entitled “SECURE SYSTEM FOR REMOVING HEAT FROM LIPID-RICH REGIONS”;
U.S. Pat. No. 8,275,442 entitled “TREATMENT PLANNING SYSTEMS AND METHODS FOR BODY CONTOURING APPLICATIONS”;
U.S. Patent Publication No. 2013/0158440 entitled “TREATMENT PLANNING SYSTEMS AND METHODS FOR BODY CONTOURING APPLICATIONS”;
U.S. patent application Ser. No. 12/275,002 entitled “APPARATUS WITH HYDROPHILIC RESERVOIRS FOR COOLING SUBCUTANEOUS LIPID-RICH CELLS”;
U.S. patent application Ser. No. 12/275,014 entitled “APPARATUS WITH HYDROPHOBIC FILTERS FOR REMOVING HEAT FROM SUBCUTANEOUS LIPID-RICH CELLS”;
U.S. Pat. No. 8,676,338 entitled “COMBINED MODALITY TREATMENT SYSTEMS, METHODS AND APPARATUS FOR BODY CONTOURING APPLICATIONS”;
U.S. Patent Publication No. 2014/0316393 entitled “COMBINED MODALITY TREATMENT SYSTEMS, METHODS AND APPARATUS FOR BODY CONTOURING APPLICATIONS”;
U.S. Pat. No. 8,603,073 entitled “SYSTEMS AND METHODS WITH INTERRUPT/RESUME CAPABILITIES FOR COOLING SUBCUTANEOUS LIPID-RICH CELLS”;
U.S. Pat. No. 9,737,434 entitled “SYSTEMS AND METHODS WITH INTERRUPT/RESUME CAPABILITIES FOR COOLING SUBCUTANEOUS LIPID-RICH CELLS”;
U.S. Pat. No. 8,702,774 entitled “DEVICE, SYSTEM AND METHOD FOR REMOVING HEAT FROM SUBCUTANEOUS LIPID-RICH CELLS”;
U.S. Pat. No. 9,861,520 entitled “DEVICE, SYSTEM AND METHOD FOR REMOVING HEAT FROM SUBCUTANEOUS LIPID-RICH CELLS”;
U.S. Patent Publication No. 2011/0300079 entitled “COMPOSITIONS FOR USE WITH A SYSTEM FOR IMPROVED COOLING OF SUBCUTANEOUS LIPID-RICH TISSUE”;
U.S. Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;
U.S. Pat. No. 6,041,787 entitled “USE OF CRYOPROTECTIVE AGENT COMPOUNDS DURING CRYOSURGERY”;
U.S. Pat. No. 6,032,675 entitled “FREEZING METHOD FOR CONTROLLED REMOVAL OF FATTY TISSUE BY LIPOSUCTION”;
U.S. Pat. No. 9,314,368 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;
U.S. Pat. No. 9,844,461 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;
U.S. Pat. No. 9,545,523 entitled “MULTI-MODALITY TREATMENT SYSTEMS, METHODS AND APPARATUS FOR ALTERING SUBCUTANEOUS LIPID-RICH TISSUE”;
U.S. Pat. No. 9,844,460 entitled “TREATMENT SYSTEMS WITH FLUID MIXING SYSTEMS AND FLUID-COOLED APPLICATORS AND METHODS OF USING THE SAME”;
U.S. Patent Publication No. 2015/0216720 entitled “TREATMENT SYSTEMS, METHODS, AND APPARATUSES FOR IMPROVING THE APPEARANCE OF SKIN AND PROVIDING FOR OTHER TREATMENTS”;
U.S. Pat. No. 9,861,421 entitled “COMPOSITIONS, TREATMENT SYSTEMS AND METHODS FOR IMPROVED COOLING OF LIPID-RICH TISSUE”;
U.S. Patent Publication No. 2015/0216719 entitled “TREATMENT SYSTEMS AND METHODS FOR TREATING CELLULITE AND FOR PROVIDING OTHER TREATMENTS”;
U.S. patent application Ser. No. 14/662,181 entitled “TREATMENT SYSTEMS, DEVICES, AND METHODS FOR COOLING TARGETED TISSUE”;
U.S. patent application Ser. No. 14/710,407 entitled “TREATMENT SYSTEMS WITH ADJUSTABLE GAP APPLICATORS AND METHODS FOR COOLING TISSUE”;
U.S. Pat. No. 9,752,856 entitled “TREATMENT SYSTEMS, SMALL VOLUME APPLICATORS, AND METHODS FOR TREATING SUBMENTAL TISSUE”;
U.S. Patent Publication No. 2016/0051308 entitled “STRESS RELIEF COUPLINGS FOR CRYOTHERAPY APPARATUSES”;
U.S. Patent Publication No. 2016/0089550 entitled “TREATMENT SYSTEMS, METHODS, AND APPARATUSES FOR ALTERING THE APPEARANCE OF SKIN”;
U.S. Patent Publication No. 2017/0007309 entitled “TREATMENT SYSTEMS AND METHODS FOR AFFECTING GLANDS AND OTHER TARGETED STRUCTURES”;
U.S. Patent Publication No. 2016/0317346 entitled “SYSTEMS AND METHODS FOR MONITORING COOLING OF SKIN AND TISSUE TO IDENTIFY FREEZE EVENTS”;
U.S. Patent Publication No. 2017/0079833 entitled “TRANSCUTANEOUS TREATMENT SYSTEMS, COOLING DEVICES, AND METHODS FOR COOLING NERVES”;
U.S. Patent Publication No. 2017/0105869 entitled “VASCULAR TREATMENT SYSTEMS, COOLING DEVICES, AND METHODS FOR COOLING VASCULAR STRUCTURES”;
U.S. Patent Publication No. 2017/0196731 entitled “TEMPERATURE-DEPENDENT ADHESION BETWEEN APPLICATOR AND SKIN DURING COOLING OF TISSUE”;
U.S. Patent Publication No. 2017/0325992 entitled “SKIN FREEZING SYSTEMS FOR TREATING ACNE AND SKIN CONDITIONS”;
U.S. Patent Publication No. 2017/0325993 entitled “HYDROGEL SUBSTANCES AND METHODS OF CRYOTHERAPY”;
U.S. Patent Publication No. 2017/0326042 entitled “LIPOSOMES, EMULSIONS, AND METHODS FOR CRYOTHERAPY”;
U.S. Patent Publication No. 2017/0326346 entitled “PERMEATION ENHANCERS AND METHODS OF CRYOTHERAPY”; and
U.S. Patent Publication No. 2017/0239079 entitled “COOLING CUP APPLICATORS WITH CONTOURED HEADS AND LINER ASSEMBLIES.”

To the extent the foregoing commonly assigned U.S. patent applications and U.S. patents or any other material incorporated herein by reference conflicts with the present disclosure, the present disclosure controls.

### BACKGROUND

Cooling treatments can be used to achieve aesthetic and/or therapeutic improvement of the human body, such as a reduction in excess adipose tissue (alternatively referred to as “body fat”). Excess adipose tissue may be present at various locations of a subject's body and may detract from personal appearance and general health. For example, excess subcutaneous fat under the chin and/or around the neck can be cosmetically unappealing and, in some instances, can produce a “double chin.” A double chin can cause stretching and/or sagging of skin and may also result in discomfort. Moreover, excess adipose tissue in superficial fat compartments can produce loose facial structures, such as loose jowls, that also cause an undesirable appearance. Excess body fat can also be located at the abdomen, thighs, buttocks, knees, and arms, as well as other locations.

Aesthetic improvement of the human body may involve the selective removal of adipose tissue. Invasive procedures (e.g., liposuction) for this purpose, however, tend to be associated with relative high costs, long recovery times, and increased risk of complications. Injection of drugs for reducing adipose tissue, such as submental or facial adipose tissue, can cause significant swelling, bruising, pain, numbness, and/or induration. Conventional non-invasive treatments for reducing adipose tissue may include regular exercise, application of topical agents, use of weight-loss drugs, dieting, or a combination of these treatments. One drawback of these non-invasive treatments is that they may not be effective or even possible under certain circumstances. For example, when a person is physically injured or ill, regular exercise may not be an option. Topical agents and orally administered weight-loss drugs are not an option if, as another example, they cause an undesirable reaction (e.g., an allergic or other negative reaction). Additionally, non-invasive treatments may be ineffective for selectively reducing specific regions of adiposity. For example, localized fat loss around the neck, jaw, cheeks, etc. often cannot be achieved using general or systemic weight-loss methods.

Furthermore, aesthetic and/or therapeutic improvement of the human body may involve treatment or alteration of non-lipid rich tissue as well as lipid rich tissue, and again conventional treatments sometimes are not suitable for many subjects and cannot effectively target certain regions of tissue necessary for an effective treatment. For at least the foregoing reasons, there is a need for innovation in this field of aesthetic and/or therapeutic improvement of the human body.

### BRIEF DESCRIPTION OF THE DRAWINGS

Many aspects of the present invention can be better understood with reference to the following drawings. The components in the drawings are not necessarily to scale. Instead, emphasis is placed on illustrating clearly the principles of the present invention. For ease of reference, throughout this disclosure identical reference numbers may be used to identify identical, similar, or analogous components or features of more than one embodiment of the present invention.

FIG. 1 is a partially cross-sectional side view of a treatment system in accordance with an embodiment of the present invention at a tissue region of a subject's body.

FIG. 2 is a flow chart illustrating a method for cooling tissue at the tissue region in accordance with an embodiment of the present invention.

FIG. 3 is a partially cross-sectional side view of a portion of the treatment system shown in FIG. 1 at the tissue region during passive diffusion of a penetration enhancer of the system into the subject's skin.

FIG. 4 is an enlarged cross-sectional view of an interface between a composite structure of the treatment system shown in FIG. 1 and the subject's skin at the tissue region during passive diffusion of the penetration enhancer into the subject's skin.

FIG. 5 is a partially cross-sectional side view of a portion of the treatment system shown in FIG. 1 at the tissue region during energy-induced diffusion of the penetration enhancer and a saccharide of the system into the subject's skin.

FIG. 6 is an enlarged cross-sectional view of the interface between the composite structure and the subject's skin at the tissue region during energy-induced diffusion of the penetration enhancer and the saccharide into the subject's skin.

FIG. 7 is an enlarged cross-sectional view of an interface between a composite structure of a treatment system in accordance with another embodiment of the present invention and the subject's skin at the tissue region during energy-induced diffusion of a saccharide of the system into the subject's skin.

FIG. 8 is a partially cross-sectional side view of an injector of a treatment system in accordance with another embodiment of the present invention at the tissue region during injection of a saccharide into the subject's skin.

FIG. 9 is an enlarged cross-sectional view of an interface between the injector shown in FIG. 8 and the subject's skin at the tissue region during injection of the saccharide into the subject's skin via a needle of the injector.

FIG. 10 is a plot of applicator temperature versus time for a cooling treatment in accordance with an embodiment of the present invention.

FIG. 11 is a chart of photographs of a subject's skin at different times following a cooling treatment with skin preparation in accordance with an embodiment of the present invention (top row) and without skin preparation (bottom row).

FIG. 12 is a plot of viscosity versus temperature for a pure saccharide and for a diluted saccharide.

### DETAILED DESCRIPTION

### Overview

In many cases, cooling treatments can be used to damage or otherwise alter certain targeted tissue while leaving non-targeted tissue near the targeted tissue undamaged or otherwise unaltered. In these cases, it may be desirable to prevent the non-targeted tissue from freezing, such as by lowering the freezing point of the non-targeted tissue and/or by suppressing nucleation of ice crystals at or near the non-targeted tissue. In addition or alternatively, it may be desirable to allow the non-targeted tissue to freeze, but to reduce the extent to which the non-targeted tissue is damaged by freezing. For example, non-targeted tissue can be exposed to an agent that helps to preserve its structures during a freeze event. Often, although not exclusively, non-targeted tissue of a cooling treatment includes skin cells. Examples of undesirable changes to a subject's skin that can result from unmitigated freeze damage include hypopigmentation, hyperpigmentation, blistering, and desquamation, among others. It may be desirable to reduce or eliminate such changes in a subject's skin in conjunction with cooling treatments that target certain subdermal tissue (e.g., subdermal lipid-rich tissue), certain dermal tissue (e.g., sebaceous cells), and/or other types of tissue for damage or other alteration.

The inventors have discovered that at least some saccharides have excellent potential for cryoprotection of non-targeted tissue during cooling treatments. Furthermore, the inventors have discovered that certain materials and processes may be beneficial in promoting diffusion of saccharides into and/or through the stratum corneum of a subject's skin to enhance cryoprotection of non-targeted tissue (e.g. skin cells). Moreover, at least some cryoprotective saccharides may provide temperature-dependent adhesive bonding that promotes stable thermal and physical contact between an applicator and a tissue region during a cooling treatment. For example, when cooled in the course of a cooling treatment, these saccharides may significantly strengthen adhesion between a subject's skin and a heat-transfer surface of an applicator, thereby reducing or eliminating relative movement between the subject's skin and the heat-transfer surface of the applicator during the cooling treatment. Further details regarding the adhesive properties of saccharides in accordance with at least some embodiments of the present invention can be found in U.S. application Ser. No. 15/400,885 entitled TEMPERATURE-DEPENDENT ADHESION BETWEEN APPLICATOR AND SKIN DURING COOLING OF TISSUE.

Cryoprotective saccharides in accordance with some embodiments of the present invention are configured to be applied as pre-treatment conditioners that begin to enhance the resistance of non-targeted tissue to cryoinjury before a cooling treatment begins. In addition or alternatively, at least some of these and/or other saccharides in accordance with embodiments of the present invention can be configured to be applied as an interface material that remains in place between an applicator and a subject's skin during a cooling treatment. Accordingly, cryoprotective saccharides in accordance with at least some embodiments of the present invention can be applied to one or more of a subject's skin, a heat transfer surface of an applicator, and an intervening structure (e.g., a liner) used with the applicator. Furthermore, at least some of these saccharides can be configured to be applied independently (e.g., as a viscous layer) or to be carried by an absorbent substrate as part of a composite structure.

In some embodiments, a method performed on a human subject having skin includes increasing a concentration of a saccharide within the subject's tissue (e.g., skin, epidermis, dermis, subcutaneous tissue, etc.). The subject's skin can be cooled via a heat-transfer surface of an applicator while the concentration of the saccharide within the subject's skin is increased a sufficient amount to inhibit, limit, or prevent thermal injury associated with the cooling. In one embodiment, a sufficient amount of the saccharide can be delivered into the tissue to enhance the tissue's resistance to cold injury while other targeted tissue is affected by the cold. An energy-delivery device can be used to apply ultrasound, optical, thermal, mechanical (e.g., vibrations), or another type of energy to the subject's skin for saccharide delivery.

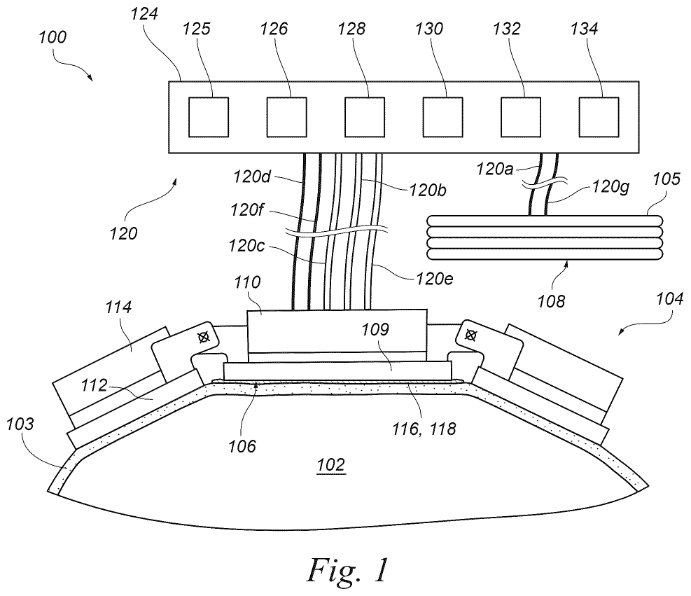

Specific details of methods for cooling tissue and related structures and systems in accordance with several embodiments of the present invention are described herein with reference to FIGS. 1-12. Although methods for cooling tissue and related structures and systems may be disclosed herein primarily or entirely in the context of cryolipolysis and cryolysis, other contexts in addition to those disclosed herein are within the scope of the present invention. For example, the disclosed methods, structures, and systems may be useful in the context of any compatible type of treatment mentioned in the applications and patents listed above and incorporated herein by reference. It should be understood, in general, that other methods, structures, and systems in addition to those disclosed herein are within the scope of the present invention. For example, methods, structures, and systems in accordance with embodiments of the present invention can have different and/or additional configurations, components, and procedures than those disclosed herein. Moreover, a person of ordinary skill in the art will understand that methods, structures, and systems in accordance with embodiments of the present invention can be without one or more of the configurations, components, and/or procedures disclosed herein without deviating from the present invention.

For ease of reference, saccharides and saccharide derivatives (i.e., modified saccharides) may be collectively referred to as “saccharides” in this disclosure. Furthermore, the term “saccharides” in this disclosure should be considered to encompass natural saccharides, artificial saccharides, and other saccharide-like polyhydroxy aldehydes and ketones. The term “treatment system,” as used generally herein, refers to cosmetic, therapeutic, or other medical treatment systems, as well as to any associated treatment regimens and medical device usages. At least some treatment systems configured in accordance with embodiments of the present invention are useful for reducing or eliminating excess adipose tissue or other undesirable tissue and/or for enhancing the appearance of skin. In many cases, the treatment systems can be used at various locations, including, for example, a subject's face, neck, abdomen, thighs, buttocks, knees, back, arms, and/or ankles. The term “tissue,” as used generally herein, may refer to a region of cells and associated extracellular material or to a type of cells and associated extracellular material.

Treatment systems in accordance with at least some embodiments of the present invention are well suited for cosmetically beneficial alterations of tissue at targeted anatomical regions. Some cosmetic procedures may be for the sole purpose of altering a target region to conform to a cosmetically desirable look, feel, size, shape, and/or other desirable cosmetic characteristic or feature. Accordingly, at least some embodiments of the cosmetic procedures can be performed without providing an appreciable therapeutic effect (e.g., no therapeutic effect). For example, some cosmetic procedures may not include restoration of health, physical integrity, or the physical well-being of a subject. The cosmetic methods can target subcutaneous or dermal regions to change a subject's appearance and can include, for example, procedures performed on subject's submental region, face, neck, ankle region, or the like. In other embodiments, however, desirable treatments may have therapeutic outcomes, such as alteration of vascular malformations, treatment of glands including sebaceous and sweat glands, treatment of nerves, alteration of body hormones levels (by the reduction of adipose tissue), etc.

Reference throughout this specification to “one example,” “an example,” “one embodiment,” or “an embodiment” means that a particular feature, structure, or characteristic described in connection with the example is included in at least one example of the present invention. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the invention. The headings provided herein are for convenience only and are not intended to limit or interpret the scope or meaning of the invention.

### Treatment Systems

FIG. 1 is a partially cross-sectional side view of a treatment system 100 in accordance with an embodiment of the present invention at a tissue region 102 of a subject's body. The treatment system 100 can include an applicator 104 (shown operably coupled to the tissue region 102) and an energy-delivery device 105 (shown separate from the tissue region 102), each configured for use at the tissue region 102. For example, the tissue region 102 can include skin 103, and the applicator 104 and the energy-delivery device 105 can be configured to be physically coupled to an outer surface of the skin 103. The applicator 104 can have a heat-transfer surface 106 through which the applicator 104 is configured to cool tissue at the tissue region 102 or to both cool and heat tissue at the tissue region 102. Similarly, the energy-delivery device 105 can have an energy-delivery surface 108 through which the energy-delivery device 105 is configured to deliver energy to tissue at the tissue region 102. In the illustrated embodiment, the applicator 104 includes a cooling element 109 coupled to a central backing 110. The applicator 104 can further include suction elements 112 coupled to respective lateral backings 114. The lateral backings 114 can be hingedly connected to the central backing 110 at opposite respective sides of the central backing 110. A strap (not shown) can be used to initially secure the applicator 104 at the tissue region 102 by compression. Suction at the suction elements 112 optionally can facilitate holding tissue at the tissue region 102 in stable contact with the cooling element 109 before cooling begins. In other embodiments, a counterpart of the applicator 104 can have another suitable form. Details regarding numerous suitable counterparts of the applicator 104 are provided in the applications and patents listed above and incorporated herein by reference, and in particular in U.S. application Ser. No. 15/400,885. These counterparts of the applicator 104 include vacuum applicators, non-vacuum applicators, “plate-type” applicators, “cup-type” applicators, “saddlebag-type” applicators, and “pinch-type” applicators, among others.

In the illustrated embodiment, the applicator 104 and the energy-delivery device 105 are configured to be used separately at the tissue region 102. In other embodiments, counterparts of the applicator 104 and the energy-delivery device 105 can be configured to be used together at the tissue region 102. Furthermore, a counterpart of the treatment system 100 can include a combined unit that serves both as an applicator and as an energy-delivery device. For example, a counterpart of the applicator 104 can be configured first to deliver heat (i.e., thermal energy) to the tissue region 102, then to remove heat from the tissue region 102, and then to again deliver heat to the tissue region 102. In at least some cases, the initial heating enhances diffusion of a cryoprotective saccharide into the tissue region 102, the subsequent cooling contributes to an aesthetic and/or therapeutic improvement of the tissue region 102, and the final heating facilitates removal of the counterpart applicator from the tissue region 102.

With reference again to FIG. 1, the treatment system 100 can further include an absorbent substrate 116 configured to be disposed between the subject's skin 103 and the applicator 104 or between the subject's skin 103 and the energy-delivery device 105. The absorbent substrate 116 can carry a saccharide (not shown) and/or a penetration enhancer (also not shown) that are also part of the treatment system 100. The saccharide can be configured to be moved into the subject's skin 103 to enhance a resistance of at least some cells within the subject's skin 103 to damage associated with cooling the subject's skin 103 via the heat-transfer surface 106 of the applicator 104. The penetration enhancer can be configured to enhance penetration of the saccharide into the subject's skin 103. Together, the absorbent substrate 116 and the material or materials it carries can form a composite structure 118. The absorbent substrate 116 can be useful, for example, to facilitate application of the saccharide and/or the penetration enhancer at low viscosities, to hold the saccharide and/or the penetration enhancer in position at the tissue region 102, to reduce or prevent displacement of the saccharide and/or the penetration enhancer during placement of the applicator 104 or during placement of the energy-delivery device 105, and/or to insure that a continuous layer of material is present between the applicator 104 and the subject's skin 103 during a cooling treatment. Insuring that a continuous layer of material is present between the applicator 104 and the subject's skin 103 during a cooling treatment can likewise insure that no part of the applicator 104 directly touches the subject's skin 103 during the cooling treatment. When supercooling temperatures are used in a cooling treatment, such direct contact between the applicator 104 and the subject's skin 103 may be undesirable as it may inadvertently inoculate the skin 103 and cause a premature freeze event therein.

In the illustrated embodiment, the absorbent substrate 116 is a generally flat, but conformable pad. In other embodiments, a counterpart of the absorbent substrate 116 can have another suitable form well suited for making optimum contact with the tissue region 102 while still being easy to apply and to remove. For example, a counterpart of the absorbent substrate 116 can be a curved pad. As another example, a counterpart of the absorbent substrate 116 can be tubular and stretchable so that it can be fitted around a subject's neck, arm, leg, torso, etc. The absorbent substrate 116 can include stretchable fabric, mesh, hydrogel, or other porous material (e.g., cotton, rayon, and polyurethane cloth) suitable for carrying the saccharide and/or the penetration enhancer. Furthermore, the absorbent substrate 116 can include a material having a relatively high thermal conductivity that at least partially compensates for a lower thermal conductivity of the material that the absorbent substrate 116 carries when the absorbent substrate 116 and the material are to be present between the applicator 104 and the subject's skin 103 during a cooling treatment. Thus, in some cases, the composite structure 118 is more thermally conductive than the material it carries. Higher thermal conductivity can be useful, for example, to facilitate detection of a thermal signature associated with a freeze event during a cooling treatment. When the absorbent substrate 116 includes stretchable fabric, some or all of the fibers of the fabric can be made of thermally conductive material. For example, the fabric can include metal fibers, carbon fibers, and/or fibers having a thermally conductive coating. Carbon fiber fabric is available, for example, under the FLEXZORB trademark from Calgon Carbon (Pittsburgh, PA).

The absorbent substrate 116 can be configured for single-use or multiple-use, and can be packaged with or without being preloaded with the saccharide and/or the penetration enhancer. When the absorbent substrate 116 is preloaded, the corresponding composite structure 118 can be encased in moisture impermeable packaging (not shown) to protect the constituent material from the environment. Furthermore, the composite structure 118 can be packaged separately from or together with a liner (also not shown) configured to protect the applicator 104 and/or the energy-delivery device 105 from the material carried by the absorbent substrate 116. In some embodiments, the composite structure 118 is pre-positioned on a liner such that the composite structure 118 and the liner can easily be brought into contact with the subject's skin 103 without any need to independently position the composite structure 118. In other embodiments, the composite structure 118 is configured to be independently placed on the subject's skin 103 and then to be pressed between the subject's skin 103 and the applicator 104. In still other embodiments, the composite structure 118 is configured to be independently placed on the subject's skin 103 and then to be removed or swapped before the applicator 104 is coupled to the subject's skin 103. While the composite structure 118 is in contact with the subject's skin 103, saccharide and/or penetration enhancer within the composite structure 118 may passively absorb into the subject's skin 103. In at least some cases, the composite structure 118 is configured to be recharged with the same or different material during and/or after this absorption.

As shown in FIG. 1, treatment system 100 can further include a support module 120 (shown schematically) and a plurality of lines 122 (individually identified as lines 122a-122g) extending between the support module 120 and the applicator 104 or between the support module 120 and the energy-delivery device 105. The support module 120 can include a housing 124 carrying an energy source 125, a fluid system 126, a power supply 128, a suction system 130, a controller 132, and an input/output device 134. The energy source 125 can be configured to drive delivery of energy (e.g., ultrasound, optical, electrical, and/or thermal energy) to tissue at the tissue region 102 via the energy-delivery device 105 before the applicator 104 is used to cool the tissue. In at least some cases, energy from the energy-delivery device 105 promotes movement of the saccharide into the subject's skin 103 thereby enhancing cryoprotection of at least some cells within the subject's skin 103. In addition or alternatively, energy from the energy-delivery device 105 may promote movement of the penetration enhancer into the subject's skin 103 thereby increasing penetration of the saccharide into the subject's skin 103 to likewise enhance cryoprotection of at least some cells within the subject's skin 103.

The fluid system 126 can be configured to chill and to circulate a heat-transfer fluid (e.g., water, glycol, or oil) through the applicator 104. For example, the fluid system 126 can include suitable fluid-cooling and fluid-circulating components (not shown), such as a fluid chamber, a refrigeration unit, a cooling tower, a thermoelectric chiller, and/or a pump. The heat-transfer fluid can be one that transfers heat with or without phase change. In some embodiments, the fluid system 126 also includes suitable fluid-heating components (also not shown), such as a thermoelectric heater configured to heat the heat-transfer fluid such that the applicator 104 can provide heating as well as cooling at the tissue region 102. In other embodiments, the treatment system 100 is configured for cooling only. The lines 122 can include an energy-delivery line 122a operably connected to the energy source 125, a supply fluid line 122b operably connected to the fluid system 126, a return fluid line 122c also operably connected to the fluid system 126, a power line 122d operably connected to the power supply 128, a suction line 122e operably connected to the suction system 130, and control lines 122f, 122g operably connected to the controller 132 and to the input/output device 134.

When in use, the treatment system 100 can deliver the heat-transfer fluid continuously or intermittently from the support module 120 to the applicator 104 via the supply fluid line 122b. Within the applicator 104, the heat-transfer fluid can circulate to absorb heat from the tissue region 102 via the heat-transfer surface 106 of the applicator 104. The heat-transfer fluid can then flow from the applicator 104 back to the support module 120 via the return fluid line 122c. For warming periods (e.g., to promote movement of a saccharide and/or a penetration enhancer into the subject's skin 103 before cooling and/or to facilitate release of the applicator 104 from the subject's skin 103), the support module 120 can actively heat the heat-transfer fluid such that warm heat-transfer fluid is circulated through the applicator 104. Alternatively or in addition, the heat-transfer fluid can be allowed to warm passively. In the illustrated embodiment, the applicator 104 relies on circulation of heat-transfer fluid to maintain a thermal gradient at an interface between the applicator 104 and the subject's skin 103 at the tissue region 102 and thereby to drive cooling or heating within the tissue region 102. In other embodiments, a counterpart of the applicator 104 can include a thermoelectric element that supplements or takes the place of circulation of heat-transfer fluid to establish and/or maintain this thermal gradient. The thermoelectric element can be configured for cooling (e.g., by the Peltier effect) and/or heating (e.g., by resistance). For example, in some embodiments, a counterpart of the applicator 104 relies on circulation of heat-transfer fluid to drive cooling and a thermoelectric element to drive heating.

The support module 120 can control the suction system 130 to apply suction via the applicator 104 and via the suction line 122e. Suction can be useful for securing a liner (not shown) to the applicator 104. Suction can also be useful for drawing in and holding the subject's skin 103 in contact with the applicator 104 or the liner during a cooling treatment. In at least some cases, the need for suction for this latter purpose is reduced or eliminated during the course of a cooling treatment due to a change in the physical properties of a saccharide disposed between the applicator 104 and the subject's skin 103. Thus, suitable suction levels can be selected based on characteristics of the tissue at the tissue region 102, patient comfort, and/or the holding power of the saccharide between the applicator 104 and the subject's skin 103. The power supply 128 can be configured to provide a direct current voltage for powering electrical elements (e.g., thermal and sensor devices) of the applicator 104 via the power line 122d. The input/output device 134 can be a touchscreen or another suitable component configured to display a state of operation of the treatment system 100 and/or a progress of a treatment protocol.

The controller 132 can be in communication with the applicator 104 and can have instructions for causing the treatment system 100 to use the applicator 104 to cool (and, in some cases, to heat) tissue at the tissue region 102. Similarly, the controller 132 can be in communication with the energy-delivery device 105 and can have instructions for causing the treatment system 100 to use the energy-delivery device 105 to promote movement of a saccharide and/or a penetration enhancer into the subject's skin 103. In at least some embodiments, the controller 132 exchanges data with the applicator 104 and/or the energy-delivery device 105 via the control lines 122f, 122g, via a wireless communication link, via an optical communication link, and/or via another suitable communications link. The controller 132 can monitor and adjust a treatment based on, without limitation, one or more treatment profiles and/or patient-specific treatment plans, such as those described in commonly assigned U.S. Pat. No. 8,275,442, which is incorporated herein by reference in its entirety. Suitable treatment profiles and patient-specific treatment plans can include one or more segments, each including a temperature profile, a vacuum level, and/or a duration (e.g., 1 minute, 5 minutes, 10 minutes, 20 minutes, 30 minutes, 1 hour, 2 hours, etc.). These treatment profiles and plans can used with any suitable applicator, such as vacuum applicators, non-vacuum applicators, “plate-type” applicators, “cup-type” applicators, “saddlebag-type” applicators, and “pinch-type” applicators, among others.

### Treatment Methods

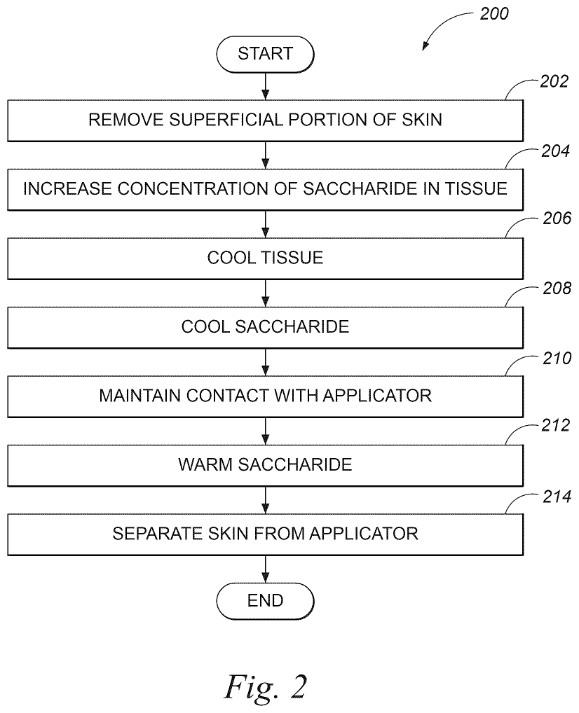

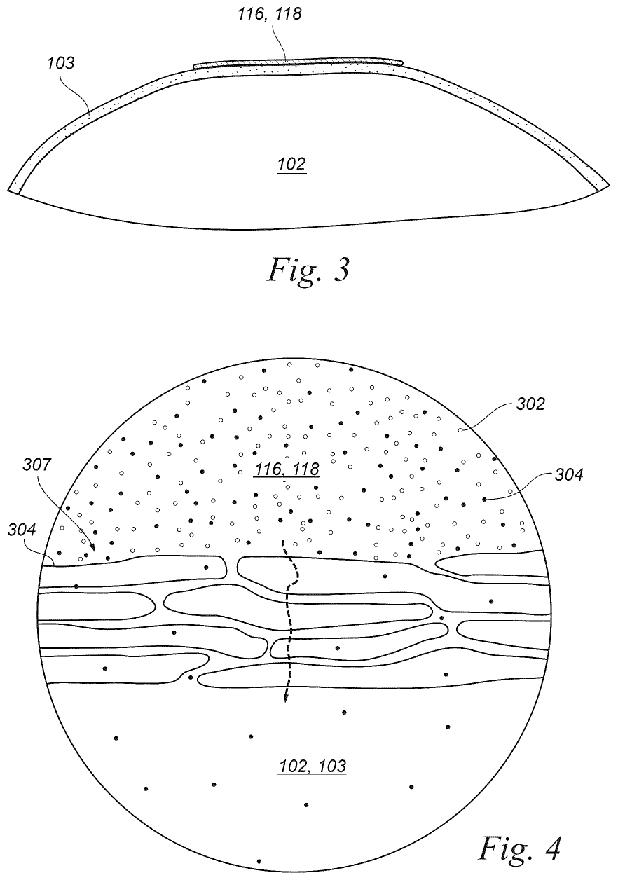

FIG. 2 is a flow chart illustrating a method 200 for cooling tissue in accordance with an embodiment of the present invention. FIGS. 3-6 illustrate the tissue region 102 and various components of the treatment system 100 during the method 200. With reference to FIGS. 1-6 together, the method 200 can begin with removal a superficial portion of the subject's skin 103 (block 202). For example, outermost corneocytes can be scraped off the subject's skin 103, pulled off the subject's skin 103 (e.g., via exfoliating tape), ablated (e.g., laser ablated), or removed in another suitable manner. This can be useful, for example, to reduce the thickness of the stratum corneum and thereby facilitate movement of a saccharide and/or a penetration enhancer through the stratum corneum.

Next, the method 200 can include increasing a concentration of the saccharide within the subject's skin 103 (block 204). For purposes of measurement, the concentration of the saccharide can be the concentration of the saccharide in the collective fluid volume of the epidermis, dermis, and subcutaneous layers of a portion of the subject's skin 103 physically and thermally coupled to the applicator 104. The increase can be from a zero concentration to a non-zero concentration, from a negligible concentration to a non-negligible concentration, from a baseline concentration to an elevated concentration, etc. Furthermore, the starting concentration can be one that provides no or only a baseline level cryoprotection, whereas the increased concentration can be one that provides a therapeutically effective elevated level of cryoprotection. In some procedures, the concentration of the saccharide is increased at least 10%, 50%, 100%, 200%, 500%, 1,000%, 1,500%, 2,000%, 3,000%, 5,000%, or more based on a desired amount of tissue protection. For example, from a starting concentration of 1 mM, the increased concentration can be at least 1.1 mM, 1.5 mM, 2 mM, 5 mM, 10 mM, 15 mM, 20 mM, 30 mM, 50 mM, or more. The starting concentration can be a normal baseline concentration or an intermediate concentration achieved (e.g., transiently achieved) while the concentration is being increased. The amount of the increase can be controlled, for example, by controlling a formulation of the saccharide, a time period during which the saccharide is allowed to diffuse into the subject's skin 103, and/or a dose of energy used to drive the saccharide into the subject's skin 103. For example, this formulation, time, energy dose, etc. can be selected to achieve a desired threshold concentration of the saccharide in the subject's skin 103 for inhibiting or substantially preventing thermal damage non-targeted tissue.

As mentioned above, increasing the concentration of the saccharide in the subject's skin 103 can include allowing the saccharide to diffuse into the subject's skin 103. This can include applying the saccharide to a surface of the subject's skin 103, such as by brushing, by smearing, by placing (e.g., when the saccharide is carried by the absorbent substrate 116), and/or by another suitable application technique. When applied, the saccharide can have a viscosity at its application temperature (e.g., room temperature, skin temperature, or body temperature) high enough to form a stable viscous layer (e.g., independently or when carried by the absorbent substrate 116) yet low enough to readily conform to irregularities (e.g., creases) in the subject's skin 103. For example, the saccharide can be applied to the subject's skin 103 at a viscosity within a range from 5,000 to 500,000 centipoise, such as within a range from 10,000 to 100,000 centipoise, from 100,000 to 200,000 centipoise, from 300,000 to 400,000 centipoise, or from 400,000 to 500,000 centipoise. In addition, when applied, the saccharide can have a low tackiness, which may substantially increase after the saccharide cools.

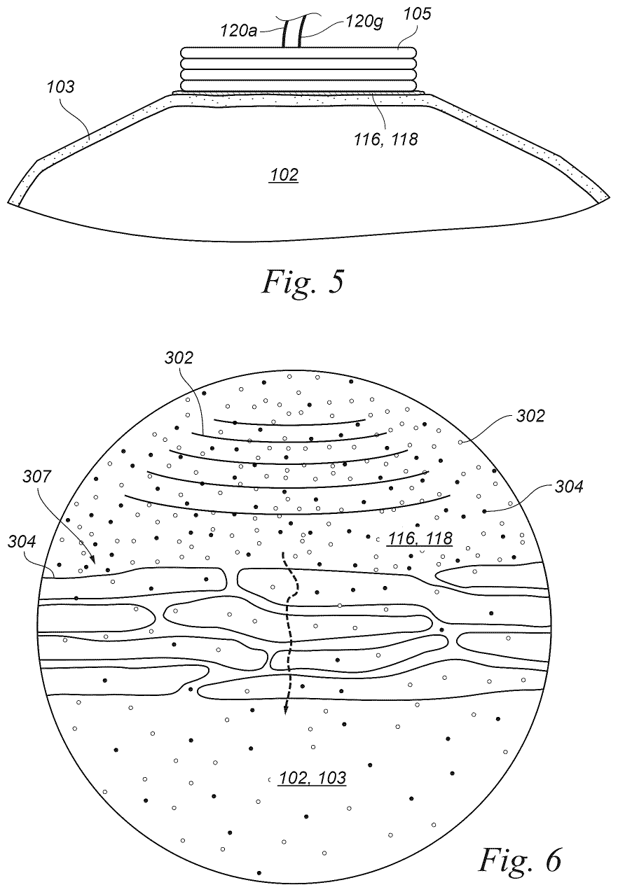

In at least some cases, the method 200 includes special features to enhance penetration of applied saccharide through the stratum corneum toward underlying cells of the subject's skin 103. FIGS. 3-6 illustrate several of these features. FIGS. 3 and 5 are partially cross-sectional side views of portions of the treatment system 100 at the tissue region 102 during passive diffusion of the penetration enhancer (FIG. 3) and during energy-induced diffusion of the penetration enhancer and the saccharide into the subject's skin 103. FIGS. 4 and 6 are enlarged cross-sectional views of the interface between the composite structure 118 and the subject's skin 103 at the tissue region 102 during the passive diffusion (FIG. 4) and the energy-induced diffusion (FIG. 6). In FIGS. 4 and 6, the saccharide and the penetration enhancer are schematically represented by unfilled circles 302 and filled circles 304, respectively. FIGS. 4 and 6 also illustrate cells 306 of the stratum corneum 307. It should be understood that the manner in which the saccharide, the penetration enhancer, and the cells 306 are represented in FIGS. 4 and 6 and elsewhere in this disclosure is a simplification that does not necessarily correspond to the actual microscopic appearance of these structures. Furthermore, it should be understood that all discussion herein of the behavior of the saccharide and the penetration enhancer within the subject's skin 103 is by way of theory, and without wishing to be bound to theory. It should also be understood that the disclosed behavior does not necessarily correspond to the actual behavior of the these substances within the subject's skin 103 in clinical practice.

With reference to FIGS. 2-4, increasing the concentration of the saccharide within the subject's skin 103 in the method 200 can include placing the absorbent substrate 116 on the surface of the subject's skin 103 while the absorbent substrate 116 carries the saccharide and the penetration enhancer. Thus, the penetration enhancer can be an excipient in a formulation carried by the absorbent substrate 116. As shown in FIG. 4, the penetration enhancer can be allowed to passively diffuse into the subject's skin 103 via transcellular and/or intercellular pathways. In some cases, the saccharide may also passively diffuse into the subject's skin 103 via these pathways, such as after the penetration enhancer has formed, enlarged, or otherwise prepared the pathways to convey the saccharide. In other embodiments, the penetration enhancer can be applied to the subject's skin 103 before the saccharide is applied. Thus, the penetration enhancer can be a conditioner rather than an excipient.

With reference to FIGS. 2, 5 and 6, increasing the concentration of the saccharide within the subject's skin 103 in the method 200 can include applying energy to the subject's skin 103 via the energy-delivery device 105 in addition to or instead of allowing the saccharide to passively diffuse into the subject's skin 103. In the illustrated embodiment, the energy is ultrasound energy represented by lines 308. In other embodiments, the method 200 can include delivering another type of energy to the subject's skin 103. For example, the method 200 can include delivering optical, electrical, and/or thermal energy to the subject's skin 103 in addition to or instead of ultrasound energy. Furthermore, the method 200 can include applying pressure (e.g., hand pressure and/or tool pressure) to the subject's skin 103 in addition to or instead of energy. The applied energy and/or pressure can promote movement of the saccharide into the subject's skin 103 by various mechanisms, such as sonophoresis (ultrasound), electroporation (electrical), and iontophoresis (also electrical), among others.

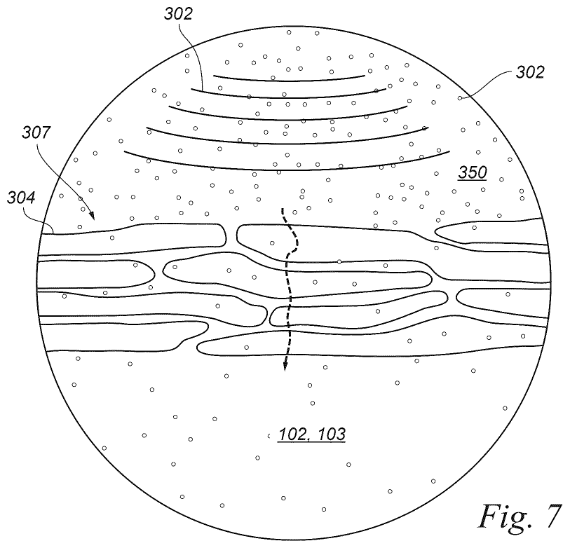

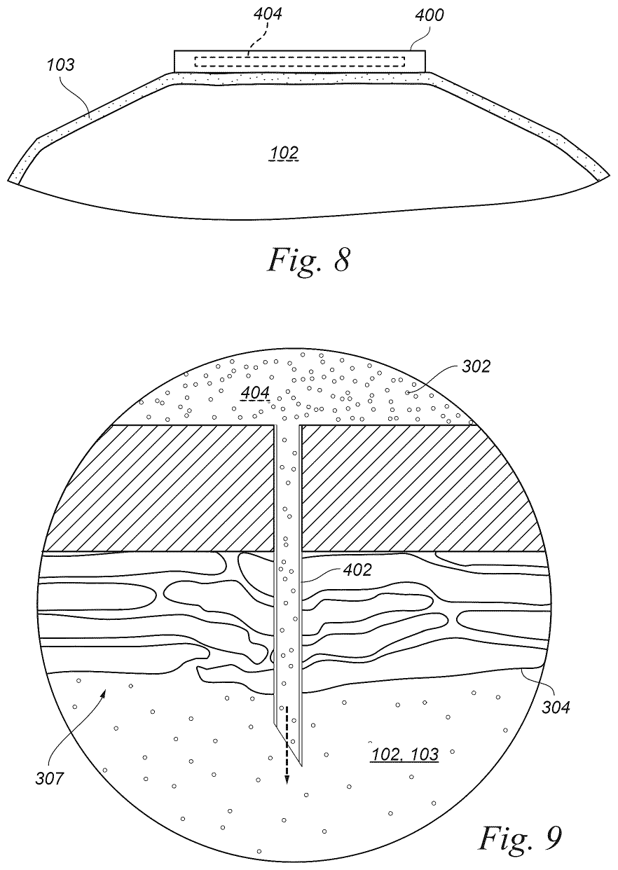

The penetration enhancer and energy delivery can be used together or separately to enhance penetration of the saccharide into the subject's skin 103. For example, FIG. 7 shows use of energy delivery without use of a penetration enhancer. Specifically, FIG. 7 is an enlarged cross-sectional view of an interface between a composite structure 350 of a treatment system in accordance with another embodiment of the present invention and the subject's skin 103 at the tissue region 102 during energy-induced diffusion of the saccharide into the subject's skin 103 in the absence of a penetration enhancer. In still other embodiments, the saccharide can be injected into the subject's skin 103. For example, FIG. 8 is a partially cross-sectional side view of an injector 400 of a treatment system in accordance with another embodiment of the present invention at the tissue region 102 during injection of the saccharide into the subject's skin 103. FIG. 9 is an enlarged cross-sectional view of an interface between the injector 400 and the subject's skin 103 at the tissue region 102 during injection of the saccharide into the subject's skin 103 via a needle 402 of the injector 400. With reference to FIGS. 8 and 9 together, the injector 400 can include a reservoir 404 containing the saccharide. Alternatively or in addition, the injector 400 can be configured to receive the saccharide from an external source (not shown) via a supply line (also not shown). As shown in FIG. 9, the needle 402 can be configured to puncture the stratum corneum 307 to create a fluid path through which the saccharide may diffuse into tissue underlying the stratum corneum 307. In at least some cases, the needle 402 is one of many microneedles of the injector 400 laterally distributed to facilitate penetration of the saccharide into the subject's skin 103 from many different points. Furthermore, the needle 402 can be a solid structure (as illustrated) or a jet.

After the concentration of the saccharide in the subject's skin 103 is increased or while the concentration of the saccharide in the subject's skin 103 is increasing, the method 200 can include cooling tissue at the tissue region 102 (block 206). For example, the method 200 can include applying the applicator 104 to the subject's skin 103, and cooling the subject's skin 103 and underlying tissue at the tissue region 102 via the heat-transfer surface 106 of the applicator 104. The saccharide within the subject's skin 103 can enhance a resistance of at least some cells within the subject's skin 103 to damage associated with the cooling. For example, the cooling can include freezing cells within the subject's skin 103, and the saccharide can enhance a resistance of the frozen cells to damage associated with the freezing. Alternatively, the cooling can include freezing cells other than the skin cells (e.g., subcutaneous lipid-rich cells), and the saccharide can prevent the skin cells from freezing along with the other cells.

In some cases, a quantity of the saccharide applied to the subject's skin 103, and that does not subsequently absorb into the subject's skin 103, is removed before the applicator 104 is used to cool the tissue region 102. Thus, the applicator 104 can be applied directly to the subject's skin 103 or coupled to the subject's skin 103 via an intervening material other than the saccharide. In other cases, a first quantity of the saccharide can be within the subject's skin 103 during the cooling, and a second quantity of the saccharide can be between the subject's skin 103 and the heat-transfer surface 106 of the applicator 104 during the cooling. For example, as shown in FIG. 1, the absorbent substrate 116 carrying the saccharide can be disposed between the heat-transfer surface 106 of the applicator 104 and the subject's skin 103 during the cooling. In these cases, the method 200 can include cooling the second quantity of the saccharide (block 208) via the heat-transfer surface 106 of the applicator 104 in conjunction with cooling the tissue region 102. Cooling the second quantity of the saccharide can reversibly strengthen an adhesive bond between the subject's skin 103 and the heat-transfer surface 106 of the applicator 104. Cooling the tissue at the tissue region 102 can occur during and/or after this strengthening of the adhesive bond.

In at least some cases, when the subject's skin 103 first moves into thermal and physical contact with the heat-transfer surface 106 of the applicator 104, the second quantity of saccharide forms a weak adhesive bond between the subject's skin 103 and the heat-transfer surface 106 of the applicator 104 such that the applicator 104 is readily repositionable before cooling begins. Repositioning the applicator 104 can be useful, for example, when an initial position of the applicator 104 is suboptimal. While cooling the tissue at the tissue region 102, the method 200 can include maintaining thermal and physical contact between the tissue and the heat-transfer surface 106 of the applicator 104 (block 210). The second quantity of the saccharide can cause this thermal and physical contact to be more reliable than it would be if the second quantity of the saccharide were not present. In at least some cases, the adhesive bond between the subject's skin 103 and the heat-transfer surface 106 of the applicator 104 may become strong enough while cooling the tissue to at least partially or totally substitute for suction and/or compression used initially to maintain the applicator 104 in contact with the tissue region 102. In these and other cases, the method 200 can include reducing or eliminating suction and/or compression after reversibly strengthening the adhesive bond and while cooling tissue at the tissue region 102. As another possible benefit, the presence of the second quantity of the saccharide during a cooling treatment may form or maintain a concentration gradient that suppresses outgoing migration of the first quantity of the saccharide, thereby prolonging a cryoprotective effect associated with the first quantity of the saccharide.

The method 200 can also include warming the second quantity of the saccharide (block 212) after cooling the second quantity of the saccharide. This can reversibly weaken the adhesion between the subject's skin 103 and the heat-transfer surface 106 of the applicator 104. In at least some embodiments, warming the second quantity of the saccharide includes warming the second quantity of the saccharide by at least 10° C. Furthermore, warming the second quantity of the saccharide can include actively warming the second quantity of the saccharide (e.g., by passing hot heat-transfer fluid through the applicator 104) and/or passively warming the second quantity of the saccharide (e.g., by passing room temperature heat-transfer fluid through the applicator 104). Warming the second quantity of the saccharide can decrease the viscosity of the second quantity of the saccharide to less than 1,000,000 centipoise. After warming the second quantity of the saccharide, the method 200 can include separating the subject's skin 103 and the heat-transfer surface 106 of the applicator 104 (block 214).

Cooling treatments in accordance with at least some embodiments of the present invention can be used to reduce or eliminate targeted tissue in either the subject's skin 103, subcutaneous layer, or other layers, and thereby cause the tissue to have a desired appearance. For example, treatment systems in accordance with embodiments of the present invention can perform medical treatments to provide therapeutic effects and/or cosmetic procedures for cosmetically beneficial effects. Without being bound by theory, the selective effect of cooling is believed to result in, for example, membrane disruption, cell shrinkage, disabling, disrupting, damaging, destroying, removing, killing, reducing, and/or other methods of lipid-rich cell and non-lipid rich cell alteration, and alteration of other tissue, either in the subject's skin 103, subcutaneous tissue, or other tissue. Such alteration is believed to stem from one or more mechanisms acting alone or in combination. It is thought that such mechanism(s) trigger an apoptotic cascade, which is believed to be the dominant form of lipid-rich cell death by non-invasive cooling. In any of these embodiments, the effect of tissue cooling can be the selective reduction of lipid-rich cells by a desired mechanism of action, such as apoptosis, lipolysis, or the like. In some procedures, an applicator 104 can cool targeted tissue of a subject to a temperature in a range of from about −25° C. to about −20° C. In other embodiments, the cooling temperatures can be from about −20° C. to about −10° C., from about −18° C. to about −5° C., from about −15° C. to about −5° C., or from about −15° C. to about 0° C. In further embodiments, the cooling temperatures can be equal to or less than −5° C., −10° C., −15° C., or in yet another embodiment, from about −15° C. to about −25° C. Other cooling temperatures and temperature ranges can be used.

Apoptosis, also referred to as “programmed cell death,” is a genetically-induced death mechanism by which cells self-destruct without incurring damage to surrounding tissues. An ordered series of biochemical events induce cells to morphologically change. These changes include cellular blebbing, loss of cell membrane asymmetry and attachment, cell shrinkage, chromatin condensation and chromosomal DNA fragmentation. Injury via an external stimulus, such as cold exposure, is one mechanism that can induce cellular apoptosis in cells. Nagle, W. A., Soloff, B. L., Moss, A. J. Jr., Henle, K. J., “Cultured Chinese Hamster Cells Undergo Apoptosis After Exposure to Cold but Nonfreezing Temperatures,” Cryobiology 27, 439-451 (1990).

One aspect of apoptosis, in contrast to cellular necrosis (a traumatic form of cell death causing local inflammation), is that apoptotic cells express and display phagocytic markers on the surface of the cell membrane, thus marking the cells for phagocytosis by macrophages. As a result, phagocytes can engulf and remove the dying cells (e.g., the lipid-rich cells) without eliciting an immune response. Temperatures that elicit these apoptotic events in lipid-rich cells may contribute to long-lasting and/or permanent reduction and reshaping of subcutaneous adipose tissue.

One mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte). Another mechanism of injury involves the lipid phase transition of those lipids within the cell's bi-lipid membrane, which results in membrane disruption or dysfunction, thereby inducing apoptosis. This mechanism is well-documented for many cell types and may be active when adipocytes, or lipid-rich cells, are cooled, Mazur, P., “Cryobiology: the Freezing of Biological Systems,” Science, 68:939-949 (1970); Quinn, P. J., “A Lipid Phase Separation Model of Low Temperature Damage to Biological Membranes,” Cryobiology, 22:128-147 (1985); Rubinsky, B., “Principles of Low Temperature Preservation,” Heart Failure Reviews, 8, 277-284 (2003).

Other possible mechanisms of adipocyte damage, described in U.S. Pat. No. 8,192,474, relate to ischemia/reperfusion injury that may occur under certain conditions when such cells are cooled as described herein. For instance, during treatment by cooling as described herein, the targeted adipose tissue may experience a restriction in blood supply and thus be starved of oxygen due to isolation as a result of applied pressure, cooling which may affect vasoconstriction in the cooled tissue, or the like. In addition to the ischemic damage caused by oxygen starvation and the buildup of metabolic waste products in the tissue during the period of restricted blood flow, restoration of blood flow after cooling treatment may additionally produce reperfusion injury to the adipocytes due to inflammation and oxidative damage that is known to occur when oxygenated blood is restored to tissue that has undergone a period of ischemia. This type of injury may be accelerated by exposing the adipocytes to an energy source (via, e.g., thermal, electrical, chemical, mechanical, acoustic, or other means) or otherwise increasing the blood flow rate in connection with or after cooling treatment as described herein. Increasing vasoconstriction in such adipose tissue by, e.g., various mechanical means (e.g., application of pressure or massage), chemical means or certain cooling conditions, as well as the local introduction of oxygen radical-forming compounds to stimulate inflammation and/or leukocyte activity in adipose tissue may also contribute to accelerating injury to such cells. Other yet-to-be understood mechanisms of injury may exist.

In addition to the apoptotic mechanisms involved in lipid-rich cell death, local cold exposure is also believed to induce lipolysis (i.e., fat metabolism) of lipid-rich cells and has been shown to enhance existing lipolysis which serves to further increase the reduction in subcutaneous lipid-rich cells. Vallerand, A. L., Zamecnik. J., Jones, P. J. H., Jacobs, I. “Cold Stress Increases Lipolysis, FFA Ra and TG/FFA Cycling in Humans” Aviation, Space and Environmental Medicine, 70, 42-50 (1999).

One expected advantage of the foregoing techniques is that the subcutaneous lipid-rich cells in the target region can be reduced generally without collateral damage to non-lipid-rich cells in the same region. In general, lipid-rich cells can be affected at low temperatures that do not affect non-lipid-rich cells. As a result, lipid-rich cells, such as those associated with highly localized adiposity (e.g., submental adiposity, submandibular adiposity, facial adiposity, etc.), can be affected while non-lipid-rich cells (e.g., myocytes) in the same generally region are not damaged. The unaffected non-lipid-rich cells can be located underneath lipid-rich cells (e.g., cells deeper than a subcutaneous layer of fat), in the dermis, in the epidermis, and/or at other locations.

In some procedures, the treatment system 100 can remove heat from underlying tissue through the upper layers of the tissue and create a thermal gradient with the coldest temperatures near the heat-transfer surface 106 of the applicator 104 (i.e., the temperature of the upper layer(s) of the subject's skin 103 can be lower than that of the targeted underlying cells). It may be challenging to reduce the temperature of the targeted cells low enough to be destructive to these target cells (e.g., induce apoptosis, cell death, etc.) while also maintaining the temperature of the upper and surface skin cells high enough so as to be protective (e.g., non-destructive). The temperature difference between these two thresholds can be small (e.g., approximately, 5° C. to about 10° C., less than 10° C., less than 15° C., etc.). Protection of the overlying cells (e.g., typically water-rich dermal and epidermal skin cells) from freeze damage during dermatological and related aesthetic procedures that involve sustained exposure to cold temperatures may include improving the freeze tolerance and/or freeze avoidance of these skin cells by using, for example, the disclosed saccharides and skin-penetration techniques. In at least some cases, the saccharides act as cryoprotectants. The saccharides and skin-penetration techniques can be used when tissue is cooled to temperatures above the freezing point of the tissue, when tissue is cooled to temperatures below the freezing point of the tissue (and when freezing does not occur due to supercooling), or when freezing of tissue is intended and caused to occur. Additional details regarding cryotherapies compatible with at least some embodiments of the present invention can be found, for example, in U.S. Patent Application Publication No. 2005/0251120, which is incorporated herein by reference in its entirety.

### Cryoprotective Saccharides

As mentioned above, the term “saccharides” in this disclosure encompasses natural and artificial saccharides as well as saccharide-like polyhydroxy aldehydes and ketones. This group includes monosaccharides, disaccharides, oligosaccharides, and polysaccharides, any of which may be useful for protecting skin cells in accordance with embodiments of the present invention. In some cases, monosaccharides and disaccharides may be preferred over oligosaccharides and polysaccharides. In some of these cases, disaccharides may be preferred over monosaccharides. By way of theory, and without wishing to be bound to theory, the cryoprotective effect of saccharides in accordance with embodiments of the present invention may be related to the properties of free aldehyde or ketone end-groups of these compounds. In particular, these end groups may bind to free amine groups of lysine and arginine in proteins of cell membranes by glycation and/or bind to polar ends of phospholipids of cell membranes by hydrogen bonding. Saccharides in accordance with at least some embodiments of the present invention bind to cell membranes more strongly than water, which the saccharides may displace. Bound saccharides may reduce or prevent cellular protein degradation during freeze/thaw procedures by confining biomolecules inside a matrix. For example, bound saccharides may form a shell around a cell membrane structure that prevents the cell membrane structure from coming into contact with another cell membrane structure and fusing. Bound saccharides may also suppress ice-crystal growth, reduce swelling, reduce osmotic shock, and/or have other cryoprotective mechanisms.

The inventors have found trehalose to be an example of a saccharide effective for reducing cryoinjury to skin cells. The inventors also expect at least some trehalose derivatives and other trehalose-like compounds to be effective for this purpose. Like trehalose, sucrose is well sized to access the phospholipid head groups of cell membranes. Accordingly, the inventors expect sucrose and at least some sucrose derivatives and other sucrose-like compounds to be effective for reducing cryoinjury to skin cells. Sucrose, however, is expected to be less able than trehalose to displace water bound to the phospholipid bilayer of cell membranes. Accordingly, trehalose may be preferred over sucrose in at least some embodiments of the present invention.

### Penetration Enhancers

The primary barrier to epidermal permeation is typically the stratum corneum. Permeation through the stratum corneum may be intercellular (i.e., through the lipid matrix between cells of the stratum corneum) or transcellular (i.e., through membranes of cells of the stratum corneum). The capacity of a molecule to enter the skin may depend on its ability to penetrate, consecutively, hydrophobic and hydrophilic barrier layers of the skin. For example, topically applied molecules may first partition into the lipophilic domain of the stratum corneum and then move into the more hydrophilic milieu of the epidermis. Therefore, molecules that penetrate well into skin may have relatively balanced lipid and water solubility. In addition, smaller (and, correspondingly, lower-molecular-weight) molecules tend to penetrate into skin more readily than larger (and, correspondingly, higher-molecular-weight) molecules. For example, cryoprotective saccharides in accordance with at least some embodiments of the present invention have a molecular weight less than 500 daltons to enhance their permeability into skin.

As described above, a penetration enhancer can be introduced into the stratum corneum to enhance permeation of a cryoprotective saccharide. Penetration enhancers may increase the permeability of skin to a cryoprotective saccharide by one or more of a variety of mechanisms including, but not limited to, extraction of lipids from the stratum corneum, alteration of the vehicle/skin partitioning coefficient, disruption of the lipid bilayer structure, displacement of bound water, loosening of horny cells, and delamination of the stratum corneum. Suitable penetration enhancers in accordance with at least some embodiments of the present invention include ethanol, polypropylene glycol, sulfoxides, laurocapram, surfactants, fatty acids, glycerol, and derivatives and combinations thereof. Furthermore, in addition to or instead of permeating into skin with assistance from a penetration enhancer, cryoprotective saccharides in accordance with at least some embodiments of the present invention can be incorporated into engineered emulsions or liposomes to facilitate skin penetration.

### Adhesion

With reference to FIG. 1, as discussed above, a first quantity of a cryoprotective saccharide in accordance with at least some embodiments of the present invention can be within a subject's skin 103 and a second quantity of the saccharide can be between the subject's skin 103 and an applicator 104 during a cooling treatment. Cooling the second quantity of the saccharide during the cooling treatment can reversibly strengthen an adhesive bond between the subject's skin 103 and the heat-transfer surface 106 of the applicator 104. Saccharides, in addition to being cryoprotective, may promote adhesion between the subject's skin 103 and the applicator 104 during a cooling treatment, for example, because they may tend to become both increasingly viscous and increasingly sticky when cooled to temperatures above their glass transition temperatures. The strength of the bond between a subject's skin 103 and the applicator 104 may benefit from both high viscosity (e.g., for maintaining the internal integrity of the bond) and high tack (e.g., for maintaining the integrity of the bonded interface between the saccharide and the skin 103).

The tendency of saccharides to become both increasingly viscous and increasingly sticky when cooled typically does not apply below their glass transition temperatures. For example, when a pure saccharide transitions to its glass state, it becomes brittle and no longer sticky. The glass transition temperatures for saccharides tend to be well above temperatures typical of cooling treatments. Saccharides in accordance with at least some embodiments of the present invention, however, are mixed with viscosity-reducing agents at ratios that move the glass-transition temperatures of the saccharides to be colder than chilled temperatures characteristic of cooling treatments in which the mixtures are to be used. In at least some cases, the glass transition temperature of a saccharide is modified in this manner such that the glass transition temperature of the corresponding mixture is colder than −20° C., such as colder than −30° C. Suitable viscosity-reducing agents include glycols (e.g., propylene glycol, dipropylene glycol, and glycerol) and other polar, biocompatible oil-like compounds. These compounds tend to be good solvents of saccharides and to have relatively low glass transition temperatures. In at least some embodiments of the present invention, a cryoprotective saccharide is mixed with a viscosity-reducing agent that also serves as a penetration enhancer.

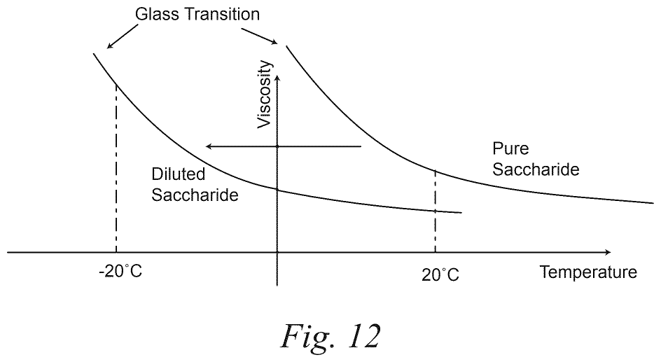

Mixing a saccharide with a viscosity-reducing agent can also be useful to modify the viscosity and/or tack temperature-dependence of the saccharide. For example, FIG. 12 is a plot of viscosity versus temperature for a pure saccharide (right) and for a saccharide diluted with a viscosity-reducing agent (left). As shown in FIG. 12, the addition of the viscosity-reducing agent lowers the glass transition temperature of the saccharide and shifts the region of highly temperature-dependent viscosity for the saccharide to be between −20° C. and 20° C. In some cases, the saccharide is a solid at room temperatures, and the viscosity-reducing agent is a liquid solvent at room temperature with a relatively high solubility limit for the saccharide, such as greater than 50% w/w, 60% w/w, 70% w/w, or a higher threshold. In other cases, the viscosity-reducing agent and the saccharide can be miscible liquids at room temperature.

The relative proportions of the saccharide and the viscosity-reducing agent in the mixture can be selected to cause a cooling temperature range in which the mixture significantly increases in viscosity and stickiness to correspond to a cooling temperature range of a treatment in which the mixture is to be used. The targeted temperature range, for example, can extend from an application temperature (e.g., room temperature, skin temperature, or body temperature) to a chilled temperature suitable for damaging or otherwise disrupting subcutaneous lipid-rich cells and/or any other targeted structures in the skin or subcutaneous layer (e.g., −20° C., −15° C., −10° C., or −5° C.). The relative proportions of the saccharide and the viscosity-reducing agent in the mixture can additionally or alternatively be selected based on the solubility limit of the saccharide in the viscosity-reducing agent. For example, the concentration of the saccharide in the mixture can be selected to be a maximum concentration (thereby maximizing the viscosity and the tack of the mixture) that still adequately suppresses recrystallization of the saccharide during normal storage and use of the mixture.

Saccharide-containing mixtures in accordance with at least some embodiments of the present invention have a viscosity less than 500,000 centipoise (e.g., within a range from 5,000 centipoise to 500,000 centipoise) at 20° C. and a viscosity greater than 3,000,000 centipoise at −15° C. In these and other cases, the viscosities of the mixtures at −10° C. can be greater than the viscosities of the mixtures at 20° C. by at least 1,000% (e.g., by at least 3,000%, 5,000%, or 10,000%) on a centipoise scale. Furthermore, the mixtures can have a first level of tensile adhesion to human skin at 20° C. and a second level of tensile adhesion to human skin at −10° C. greater that the first level of tensile adhesion by a factor of more than 1.25×, 1.5×, 2×, 3×, 4×, 5×, 6×, 7×, 10×, 20×, or 30×. This tensile adhesion to human skin can be tested by applying a normal pulling force to a flat layer of the mixture disposed between an applicator and a skin analog.

### Experimental Example

A mixture of trehalose, water, and polypropylene glycol (PG) was tested as a pre-cooling skin treatment. The mixture was formed by combining 100 grams of trehalose, 100 mL of water, and 40 mL of PG. The trehalose was expected to provide cryoprotection, while the water and PG were expected to act as penetration-enhancing excipients. A first site at a subject's right flank was given the pre-cooling skin treatment, while an opposite second site at the subject's left flank was designated as a control. The pre-cooling treatment at the first site included placing a small piece of rayon fabric soaked in 100 mL of the mixture directly onto the subject's skin and waiting for 12 minutes. It was expected that passive diffusion of trehalose into the subject's skin occurred to some extent during this period. Next, an ultrasound cavitation device (40 kHz) was moved top-to-bottom and bottom-to-top over the subject's skin at the first site for 3 minutes. During this period, 5 mL of the mixture was poured over the subject's skin at the first site.

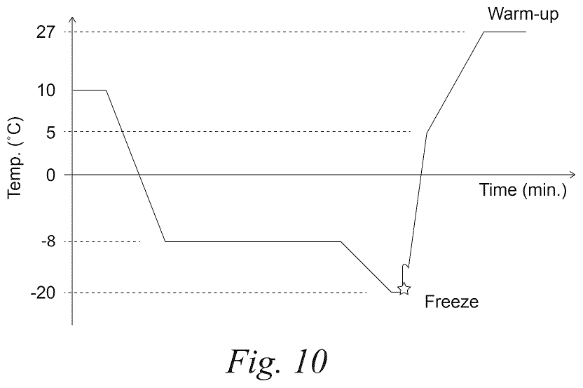

After the pre-cooling skin treatment at the first site, an applicator having a rectangular plate-type heat-transfer surface with an area of 1.7 square inches was used to execute a cooling treatment at the first and second sites. During the cooling treatment, a water-based hydrogel was disposed between the heat-transfer surface of the applicator and the subject's skin. The cooling treatment was set to supercool the skin for 2 minutes at −8° C., decrease the temperature of the skin until a freeze occurs, hold the skin in a frozen state for about 45 seconds, and then rapidly warm the skin. FIG. 10 is a plot of applicator temperature versus time for the cooling treatment. When subjected to the cooling treatment, the first site achieved a lowest temperature of −12° C. and maintained a frozen state for 47 seconds. When subjected to the cooling treatment, the second site achieved a lowest temperature of −11.5° C. and maintained a frozen state for 48 seconds.

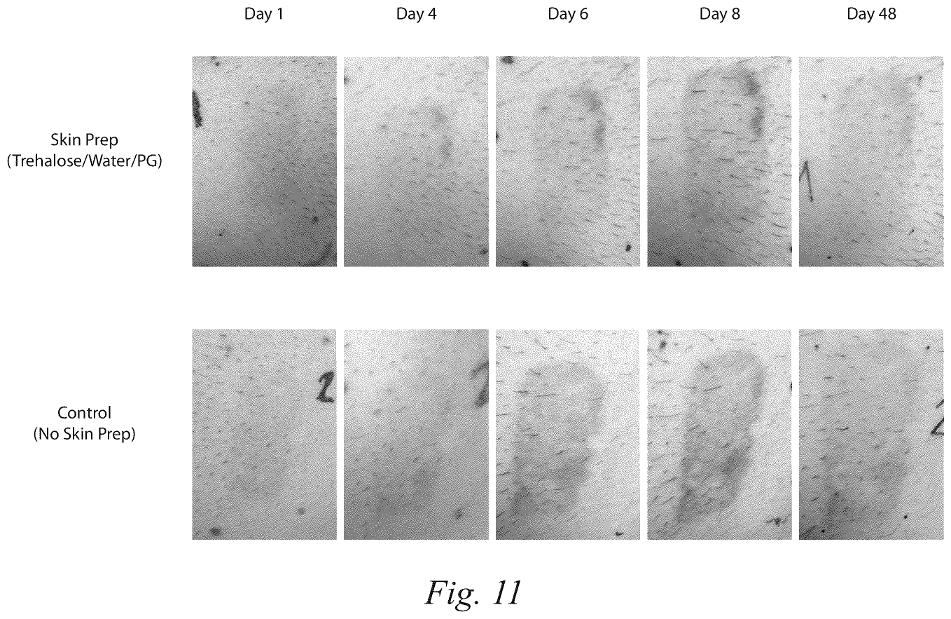

FIG. 11 is a chart of photographs of the first site (top row) and the second site (bottom row) at different times following the cooling treatment. As shown in FIG. 11, the pre-cooling skin treatment considerably reduced erythema after 8 days and hyperpigmentation after 48 days. In view of FIG. 11, the inventors have demonstrated that cryoprotection using a mixture of trehalose, water and propylene glycol diminishes immediate freezing damage (observed as reduced erythema) and lessens hyperpigmentation after a freeze insult when compared to a control site.

### CONCLUSION

Various embodiments of the invention are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the invention, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above. While processes or acts are presented in a given order, alternative embodiments may perform the processes or acts in a different order, and some processes or acts may be modified, deleted, and/or moved. The headings provided herein are for convenience only and do not interpret the scope or meaning of the described invention.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.).

Any patents, applications and other references, including any that may be listed in accompanying filing papers, are incorporated herein by reference. Aspects of the described invention can be modified, if necessary, to employ the systems, functions, and concepts of the various references described above to provide yet further embodiments. These and other changes can be made in light of the above Detailed Description. While the above description details certain embodiments and describes the best mode contemplated, no matter how detailed, various changes can be made. Implementation details may vary considerably, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

I/We claim:

1. A method performed on a human subject having skin, the method comprising:
increasing a concentration of a modified or unmodified saccharide within the subject's skin;
applying an applicator to the subject's skin; and
cooling the subject's skin via a heat-transfer surface of the applicator while the concentration of the saccharide within the subject's skin is increased, wherein the saccharide within the subject's skin enhances a resistance of at least some cells within the subject's skin to damage associated with the cooling.

2. A system for use on a human subject having skin, the system comprising
an applicator having a heat-transfer surface, wherein the applicator is configured to cool the subject's skin via the heat-transfer surface; and
a modified or unmodified saccharide configured to be disposed within the subject's skin to enhance a resistance of at least some cells within the subject's skin to damage associated with cooling the subject's skin via the heat-transfer surface of the applicator.

3. The system of claim 2 wherein:
the saccharide is configured to be applied to a surface of the subject's skin; and
the system further comprises an energy-delivery device configured to drive at least some of the applied saccharide into the subject's skin.

4. The system of claim 3 wherein the energy-delivery device is configured to apply ultrasound energy to the subject's skin to drive at least some of the applied saccharide into the subject's skin.

5. The system of claim 3 wherein the energy-delivery device is configured to apply optical energy to the subject's skin to drive at least some of the applied saccharide into the subject's skin.

6. The system of claim 3 wherein the energy-delivery device is configured to apply thermal energy to the subject's skin to drive at least some of the applied saccharide into the subject's skin.

7. The system of claim 2, further comprising an injector configured to inject the saccharide into the subject's skin.

8. The system of claim 2 wherein:
the saccharide is configured to be applied to a surface of the subject's skin; and
the system further comprises a penetration enhancer configured to enhance penetration of the saccharide into the subject's skin.

9. The system of claim 8 wherein the penetration enhancer is configured to be applied to the subject's skin before the saccharide is applied to the subject's skin.

10. The system of claim 8 wherein the penetration enhancer and the saccharide are configured to be applied to the subject's skin simultaneously.

11. The system of claim 8 wherein the penetration enhancer is selected from a group consisting of ethanol, polypropylene glycol, sulfoxides, laurocapram, surfactants, fatty acids, glycerol, and derivatives and combinations thereof.

12. The system of any of claim 2 wherein the saccharide is a disaccharide or a derivative thereof.

13. The system of any of claim 2 wherein the saccharide is trehalose or a derivative thereof.

14. The system of any of claim 2 wherein the saccharide is sucrose or a derivative thereof.

15. The system of any of claim 2 wherein:
the saccharide within the subject's skin is a first quantity of the saccharide;
the system further comprises a second quantity of the saccharide configured to be disposed between the subject's skin and the heat-transfer surface of the applicator while the applicator cools the subject's skin via the heat-transfer surface; and
the second quantity of the saccharide is configured to reversibly increase tensile adhesion between the subject's skin and the heat-transfer surface of the applicator by a factor of at least 1.25 while the applicator cools the subject's skin via the heat-transfer surface.

16. The system of claim 15 wherein the second quantity of the saccharide is configured to reversibly increase tensile adhesion between the subject's skin and the heat-transfer surface of the applicator by a factor of at least 2 while the applicator cools the subject's skin via the heat-transfer surface.

17. The system of claim 15 wherein:
the second quantity of the saccharide is configured to cool without undergoing a glass transition while the applicator cools the subject's skin via the heat-transfer surface; and
the second quantity of the saccharide is configured to increase in viscosity by at least 1,000% on a centipoise scale while the applicator cools the subject's skin via the heat-transfer surface.

18. The system of claim 15 wherein:
the second quantity of the saccharide is configured to cool without undergoing a glass transition while the applicator cools the subject's skin via the heat-transfer surface; and
the second quantity of the saccharide is configured to increase in viscosity by at least 10,000% on a centipoise scale while the applicator cools the subject's skin via the heat-transfer surface.

19. The system of claim 15, further comprising an absorbent substrate configured carry the second quantity of the saccharide and to be disposed between the heat-transfer surface of the applicator and the subject's skin while the applicator cools the subject's skin via the heat-transfer surface.
